(19) United States
(12) Patent Application Publication
(10) Pub. No.: US 2004/0234572 A1
(43) Pub. Date: Nov. 25, 2004

(54) Preparation of sustained release pharmaceutical composition

(76) Inventors: Serge R. Martinod, Groton, CT (US); Malcolm Brandon, Bulleen (AU); Robert V. Packard, Wetherfield, CT (US)

Correspondence Address: MORGAN LEWIS & BOCKIUS LLP, 1111 PENNSYLVANIA AVENUE NW, WASHINGTON, DC, 20004, US

(21) Appl. No.: 10/487,714

(22) PCT Filed: Jul. 1, 2002
(86) PCT No.: PCT/AU02/00868

(30) Foreign Application Priority Data
Sep. 11, 2001 (AU) PR7614

(51) Int. Cl. A61F 2/00

(52) U.S. Cl. 424/423

(57) ABSTRACT

A sustained release apparatus including a silicone support material; and a pharmaceutically active composition carried in or on the silicone support material; the pharmaceutically active composition including at least one pharmaceutically active component; and optionally a carrier therefor; the pharmaceutically active component being present in amounts of from approximately 30% to 75% by weight, based on the total weight of the sustained release apparatus.

The present invention relates to sustained release pharmaceutical compositions, and in particular a method for the preparation thereof. More specifically, the present invention relates to a sustained release pharmaceutical composition, which provides a significant increase in pharmaceutical payload.

A number of drug delivery systems are known in the prior art.

For example, a controlled drug-release preparation using as a carrier a hydrophobic polymer material, which is nondegradable after administration into the living body. There are two methods of controlling release of a drug from such preparation; one, using an additive such as an albumin (Japanese patent publication (Tokkohei) No. 61959/1995), and another, by forming an outer layer consisting of hydrophobic polymer alone (Japanese patent publication (Tokkaihei) No. 187994/1995).

However, where a disease indication requires the achievement of a high threshold blood plasma level and/or requires the delivery of multiple pharmaceuticals and/or requires sustained release to be continued over an extended period at high levels, the drug delivery systems known in the prior art generally exhibit insufficient drug carrying capacity.

In addition, techniques known in the prior art for producing sustained release implants utilise a silicone based technology based on an extrusion or molding system.

Difficulties have been encountered in attempting to scale up such techniques to commercial volumes. Difficulties have also been encountered in applying such extrusion techniques to pharmaceutical actives such as Ceftiofur and Recombinant Porcine Somatotropin (rPST). For example, such activities interfere with silicone chemistry due to their chemical composition or exhibit temperature sensitivity.

It is, accordingly, an object of the present invention to overcome or at least alleviate one or more of the difficulties and deficiencies related to the prior art.

Accordingly, in a first aspect of the present invention, there is provided a sustained release apparatus including

a silicone support material; and

a pharmaceutically active composition carried in or on the silicone support material;

the pharmaceutically active composition including

at least one pharmaceutically active component; and optionally

a carrier therefor;

the pharmaceutically active component being present in amounts of from approximately 30% to 75% by weight, based on the total weight of the sustained release apparatus.

The sustained release apparatus is preferably of the form of an uncovered or covered rod or dispersed matrix type. Whilst such apparatuses have been proposed in the prior art, such apparatuses in the prior art have been limited by their ability to provide relatively low loading capacities, e.g. less than 30% by weight of active.

The sustained release apparatus according to the present invention preferably exhibits loading capacities of pharmaceutical active of 35% to 65% by weight, more preferably 35% to 55% by weight, most preferably approximately 40% to 50% by weight, based on the total weight of the apparatus.

Such increased loading capacity permits the treatment of diseases over an extended period with pharmaceutically active components which have heretofore not been applicable to such diseases as it has not been possible to achieve the required threshold blood plasma levels to be efficacious and to maintain those blood levels over an extended period of time.

Preferably the sustained release apparatus may provide approximately zero order release of pharmaceutical active.

For example, in veterinary applications, the pharmaceutically active component ivermectin is a mixture of not less than 90% ivermectin H₂B₁a and not more than 5% ivermectin H₂B₁b having the respective molecular weights 875.10 and 861,07. Ivermectin is a potent macrocyclic lactone disaccharide antiparasitic agent used to prevent and treat parasite infestations in animals. The compound has activity against both internal and external parasites as well as being effective against arthropods, insects, nematodes, filarioidea, platyhelminths and protozoa.

Other macrocyclic lactones which may be used include moxidectin, eprliomectin, doramectin or mixtures thereof.

Accordingly, in a preferred aspect there is provided a sustained release apparatus including

a silicone support material; and

an anthelmintic composition carried in or on the support material;

the anthelmintic composition including

an anthelmintic component; and optionally

a carrier therefor;

the anthelmintic component being present in amounts of from approximately 30% to 75% by weight, preferably approximately 35% to 55% by weight, more preferably approximately 40% to 50% by weight, based on the total weight of the apparatus.

The anthelmintic component preferably includes a macrocyclic lactone, more preferably ivermectin.

The silicone support material may be formed from a silicone elastomer. The silicone support material may include a liquid silicone as described below.

The anthelmintic carrier, when present, may include standard carrier components as described below.

The silicone support material may be of any suitable form. The sustained release support material may take the form of a support matrix or rod, preferably a covered rod structure.

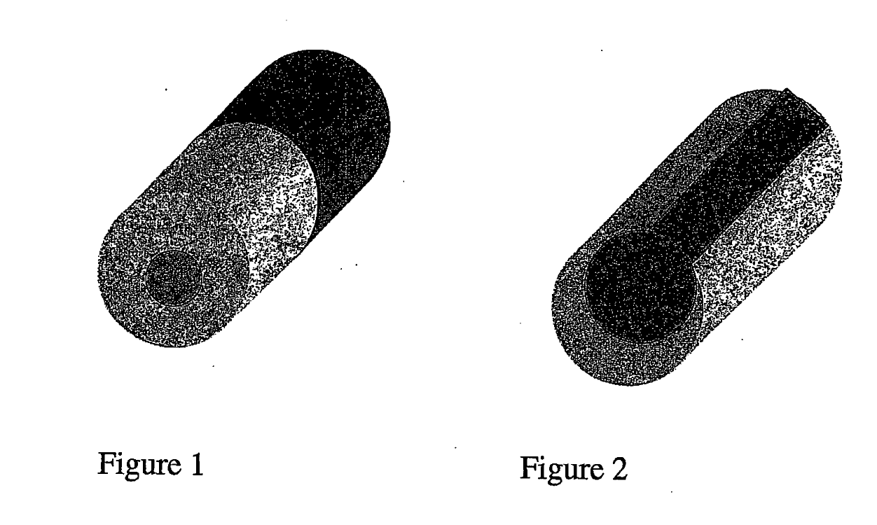

A partially covered rod may be used. Such a structure permits further modification of the release characteristics of the sustained release apparatus according to the present invention. An eccentric or asymmetric rod, optionally partially or fully covered, may be used. Illustrative examples thereof are provided in FIGS. 1 and 2 below.

In a preferred aspect of the present invention the sustained release apparatus may take the form of a biocompatible article suitable for insertion into the body of an animal to be treated.

The biocompatible article may include a medical instrument, apparatus or prosthetic device, or part thereof.

Accordingly, in this aspect of the present invention, there is provided a biocompatible article including a sustained release apparatus including

a silicone support material; and

a pharmaceutically active composition carried in or on the silicone support material;

the pharmaceutically active composition including

at least one pharmaceutically active component, and optionally

a carrier therefor;

the pharmaceutically active component being present in amounts of from approximately 30% to 75% by weight, preferably approximately 35% to 65% by weight, based on the total weight of the sustained release apparatus.

For example, the biocompatible article may include a catheter, or prosthetic appliance, or medical implant, e.g. for reconstructive, dental or cosmetic surgery. Implant materials for replacing or filling bone or like defects are particularly preferred.

It will be understood that by incorporating a pharmaceutically active composition in or on such biocompatible articles, a sustained therapeutic effect may be achieved at the site of insertion.

For example, heparin, an anti-coagulation agent, may be included as the pharmaceutically active component on, or in, e.g a catheter, thus reducing the possibility of blood clots during surgical or other medical procedures.

Similarly, verapamil, an anti-anginal agent, may be included in biocompatible article such as synthetic heart valves, arterial implants, or part thereof, or the like as a prophylactic treatment against anginal attacks.

Growth factors, e.g. nerve growth factors, may similarly be included, for example to assist the healing process, e.g. after surgical procedures.

In the sustained release apparatus according to the present invention, the silicone support material may be formed from a silicone base polymer. The silicone base polymer may be of any suitable type. A biocompatible silicone base polymer is preferred. A methyl-vinyl polysiloxane polymer is preferred. A vinyl-substituted dimethyl siloxane polymer is particularly preferred. A low viscosity material is preferred, particularly for extrusion applications. A 40-durometer or lower formulation is preferred.

A reinforcing filler, e.g. a silica, preferably a fumed silica, may be included in the silicone base polymer. A silicone elastomer including fumed silica sold under the trade designations CS10401 or CS10701, and blends thereof, available from IMMIX Technologies LLC, Cri-Sil Division, have been found to be suitable. The reinforcing filler may be present in amounts of from approximately 1.0 to 33% by weight, preferably 10 to 20%, more preferably 10 to 15% by weight, based on the total weight of the sustained release apparatus.

The silicone base polymer component may be present in amounts of from approximately 15 to 70% by weight, preferably approximately 25% to 65% by weight, based on the total weight of the apparatus. The silicone base polymer can be either liquid form or “gum stock.” Preference is dictated by the type of process used to form and coat the sustained release apparatus. Blending of multiple forms is a typical procedure for obtaining the desired physical properties.

The pharmaceutically active composition, as described above, may include

at least one pharmaceutically active component; and optionally

a carrier therefor.

The pharmaceutically active component may include a water-insoluble pharmaceutical, a water-soluble pharmaceutical, a lipophilic pharmaceutical, or mixtures thereof.

The pharmaceutically active component may be exemplified by, but not limited to, one or more selected from the group consisting of:

| | |
|---|---|
| Acetonemla preparations | Anabolic agents |
| Anaesthetics | Analgesics |
| Anti-acid agents | Anti-arthritic agents |
| Antibodies | Anti-convulsivants |
| Anti-fungals | Anti-histamines |
| Anti-infectives | Anti-Inflammatories |
| Anti-microbials | Anti-parasitic agents |
| Anti-protozoals | Anti-ulcer agents |
| Antiviral pharmaceuticals | Behaviour modification drugs |
| Biologicals | Blood and blood substitutes |
| Bronchodilators and expectorants | Cancer therapy and related |
| | pharmaceuticals |
| Cardiovascular pharmaceuticals | Central nervous system |
| | pharmaceuticals |
| Coccidiostats and coccidiocidals | Contraceptives |
| Contrast agents | Diabetes therapies |
| Diuretics | Fertility pharmaceuticals |
| Growth hormones | Growth promoters |
| Hematinics | Hemostatics |
| Hormone replacement therapies | Hormones and analogs |
| Immunostimulants | Minerals |
| Muscle relaxants | Natural products |
| Nutraceuticals and nutritionals | Obesity therapeutics |
| Ophthalmic pharmaceuticals | Osteoporosis drugs |
| Pain therapeutics | Peptides and polypeptides |
| Respiratory pharmaceuticals | Sedatives and tranquilizers |
| Transplantation products | Urinary acidifiers. |
| Vaccines and adjuvants | Vitamins |

The pharmaceutically active component may include a water-insoluble pharmaceutical, a water-soluble pharmaceutical, a lipophilic pharmaceutical or mixtures thereof.

The pharmaceutically active component may be a heat-susceptible component such as rPST and/or a sulfur-containing component such as ceftiofur.

The water-soluble pharmaceuticals useful in the sustained release apparatus according to the present invention include such drugs as peptides, polypeptides, proteins, glycoproteins, polysaccharides, and nucleic acids.

The present invention is particularly appropriate for pharmaceuticals that are very active even in extremely small quantities and whose sustained long-term administration is sought. When used in substantially increased quantities, such pharmaceuticals may be applied to disease indications heretofore untreatable over an extended period. The pharmaceuticals may be exemplified by, but not limited to, one or more selected from the group consisting of cytokines (eg. interferons and interleukins), hematopoietic factors (eg. colony-stimulating factors and erythropoietin), hormones (eg. growth hormone, growth hormone releasing factor, calcitonin, leuteinizing hormone, leuteinizing hormone releasing hormone, and insulin), growth factors (eg. somatomedin, nerve growth factor), neurotrophic factors, fibroblast growth factor, and hepatocyte proliferation factor; cell adhesion factors; immunosuppressants; enzymes (eg. asparaginase, superoxide dismutase, tissue plasminogen activating factor, uroinase, and prourokinase), blood coagulating factors (eg. blood coagulating factor VIII), proteins involved in bone metabolism (eg. BMP (bone morphogenetic protein)), and antibodies.

The interferons may include alpha, beta, gamma, or any other interferons or any combination thereof. Likewise, the interleukin may be IL-1, IL-2, IL-3, or any others, and the colony-stimulating factor may be mufti-CSF (multipotential CSF), GM-CSF (granulocyte-macrophage CSF), G-CSF (granulocyte CSF), M-CSF (macrophage CSF), or any others.

Vaccines are particularly preferred. The vaccines useful in the sustained release apparatus according to the present invention may be exemplified by, but not limited to, one or more selected from the group consisting of vaccines against

| | |
|---|---|
| Adenovirus | Anthrax |
| BCG | Chlamydia |
| Cholera | Circovirus |
| Classical swine fever | Coronavirus |
| Diphtheria-Tetanus (DT for children) | Diphtheria-Tetanus (tD for adults) |
| Distemper virus | DTaP |
| DTP | E coli |
| Elmeria (coccidosis) | Feline immunodeficiency virus |
| Feline leukemia virus | Foot and mouth disease |
| Hemophilus | Hepatitis A |
| Hepatitis B | Hepatitis B/Hib |
| Herpes virus | Hib |
| Influenza | Japanese Encephalitis |
| Lyme disease | Measles |
| Measles-Rubella | Meningococcal |
| MMR | Mumps |
| Mycoplasma | Para influenza virus |
| Parvovirus | Pasteurella |
| Pertussis | Pestivirus |
| Plague | Pneumococcal |
| Pollo (IPV) | Polio (OPV) |
| Pseudorables | Rabies |
| Respiratory syncitial virus | Rotavirus |
| Rubella | Salmonella |
| Tetanus | Typhoid |
| Varicella | Yellow Fever |

Pharmaceuticals that can be applied in pharmaceutically active compositions according to the present invention may be further exemplified by low-molecular-weight drugs such as water-soluble anticancer agents, antibiotics, anti-inflammatory drugs, alkylating agents, and immunosuppressants. Examples of these drugs include adriamycin, bleomycins, mitomycins, fluorouracil, peplomycin sulfate, daunorubicin hydrochloride, hydroxyurea, neocarzinostatin, sizofiran, estramustine phosphate sodium, carboplatin, beta actams, tetracyclines, aminoglycosides, and phosphomycin.

The pharmaceutically active composition of the present invention may contain two or more drugs depending on the disease and method of application.

For example, in veterinary applications for control of parasitic infections, a combination of ivermectin and praziquantel or a combination of zeranol and trembolone may be used.

Water-insoluble pharmaceutically active components which may be utilised in the sustained release apparatus according to the present invention include lipophilic pharmaceuticals.

A lipophilic pharmaceutical may be any lipophilic substance so long as it is, as a form of a preparation, in a solid state at the body temperature of an animal or a human being to which the preparation is to be administered. Lipophilic as herein used means that the solubility of a substance in water is low, which specifically includes the following natures, as described in Pharmacopoeia of Japan 13th Edition (1996): practically insoluble (the amount of more than or equal to 10000 ml of solvent is required to dissolve 1 g or 1 ml of a solute), very hard to dissolve (the amount of more than or equal to 1000 ml and less than 10000 ml of solvent is required to dissolve 1 g or 1 ml of a solute), or hard to dissolve (the amount of more than or equal to 100 ml and less than 1000 ml of solvent is required to dissolve 1 g or 1 ml of a solute).

Specific examples of the lipophilic pharmaceutical include, but are not limited to, antibiotics such as avermectin, ivermectln, spiramycin, and ceftiofur; antimicrobials (eg. amoxicillin, erythromycin, oxytetracycline, and lincomycin), anti-inflammatory agents (eg. dexamethasone and phenylbutasone), hormones (eg. levothyroxine), adrenocorticosterolds (eg. dexamethasone palmitate, triamcinolone acetonide, and halopredone acetate), non-steroidal anti-inflammatory agents (eg. indometacin and aspirin), therapeutic agents for arterial occlusion (eg. prostaglandin E1), anticancer drugs (eg. actinomycin and daunomycin), therapeutic agents for diabetes (eg. acetohexamide), and therapeutic agents for osteopathy (eg. estradiol).

Depending on a disease or a method for application, multiple lipophilic drugs may be contained. In addition to the lipophilic drug having a direct therapeutic effect, the drug may be a substance with a biological activity, and such a substance as promotes or induces a biological activity, which includes an adjuvant for a vaccine, for example saponin. In such a case, incorporation of a vaccine into a preparation results in a sustained release preparation of a vaccine with an adjuvant.

The pharmaceutically active composition is characterised by including an amount of pharmaceutical active component up to approximately 85% by weight, preferably less than approximately 75% by weight, based on the total weight of the sustained release apparatus.

As stated above, the pharmaceutically active composition according to the present invention may further include a carrier for the pharmaceutically active component.

The pharmaceutical carrier may be selected to permit release of the pharmaceutically active component over an extended period of time from the composition.

The carrier may include a water-soluble substance.

A water-soluble substance is a substance which plays a role of controlling infiltration of water into the inside of the drug dispersion. There is no restriction in terms of the water-soluble substance so long as it is in a solid state (as a form of a preparation) at the body temperature of an animal or human being to which it is to be administered, and a physiologically acceptable, water-soluble substance.

One water-soluble substance, or a combination of two or more water-soluble substances may be used. The water-soluble substance specifically may be selected from one or more of the group consisting of synthetic polymers (eg. polyethylene glycol, polyethylene polypropylene glycol), sugars (eg. sucrose, mannitol, glucose) sodium chondroitin sulfate, polysaccharides (e.g. dextran) amino acids (eg. glycine and alanine), mineral salts (eg. sodium chloride), organic salts (eg. sodium citrate) and proteins (eg. gelatin and collagen and mixtures thereof). A sugar or salt or mixtures thereof are preferred. A mixture of sodium chloride and mannitol is particularly preferred.

In addition, when the water-soluble substance is an amphipathic substance, which dissolves in both an organic solvent and water, it has an effect of controlling the release of, for example, a lipophilic drug by altering the solubility thereof. An amphipathic substance includes, but is not limited to, polyethylene glycol or a derivative thereof, polyoxyethylene polyoxypropylene glycol or a derivative thereof, a fatty acid ester, a sodium arlylsulfate of sugars, and more specifically, polyethylene glycol, polyoxy stearate 40, polyoxyethylene[196]polyoxypropylene [67]glycol, polyoxyethylene[105]polyoxypropylene[5]glycol, polyoxyethylene[160]-polyoxypropylene[30]glycol, sucrose esters of fatty acids, sodium lauryl sulfate, sodium oleate, and sodium desoxycholic acid (sodium deoxycholic acid (DCA)).

Polyoxyethylene polyoxypropylene glycol (also called poloxymers as a generic term), sucrose, or a mixture of sucrose and sodium deoxycholic acid (DCA) are preferred.

In addition, the water-soluble substance may include a substance which is water-soluble and has any activity in vivo, such as low molecular weight drugs, peptides, proteins, glycoproteins, polysaccharides, or antigenic substances used as vaccines, i.e. water-soluble drugs.

The pharmaceutical carrier may constitute from approximately 0% to 30% by weight, preferably approximately 15% to 25% by weight, more preferably approximately 10% to 20% by weight, based on the total weight of the sustained release apparatus.

The sustained release apparatus may include additional carrier or excipients, fillers, lubricants, plasticisers, binding agents, pigments and stabilising agents.

Suitable fillers may be selected from the group consisting of talc, titanium dioxide, starch, kaolin, cellulose (microcrystalline or powdered) and mixtures thereof.

Where the sustained release apparatus takes the form of a biocompatible article, e.g. an implant, calcium fillers, e.g. calcium phosphate, are particularly preferred.

Suitable binding agents include polyvinyl pyrrolidine, hydroxypropyl cellulose and hydroxypropyl methyl cellulose and mixtures thereof.

Accordingly, in a further aspect of the present invention, there is provided a process for the preparation of a sustained release apparatus including

a silicone support material; and

a pharmaceutically active composition carried in or on the silicone support material;

the pharmaceutically active composition including

at least one pharmaceutically active component; and optionally

a carrier therefor;

the pharmaceutically active component being present in amounts of from approximately 30% to 75% by weight, based on the total weight of the sustained release apparatus, which process includes

providing

a silicone base polymer;

a cross-inking agent;

a pharmaceutically active component;

a peroxide or metal catalyst; and

a low temperature curing inhibitor;

pre-mixing at least a portion of the silicone base polymer and the metal catalyst together to form a first part;

pre-mixing the cross-linking agent, low temperature curing inhibitor, any remaining silicone base polymer, and pharmaceutical active for a time sufficient to at least partially wet the pharmaceutical active and form a second part; and

mixing the first and second parts together as a batch or continuously; and

feeding the mixture into a molding or extrusion apparatus at a relatively low temperature for a relatively short time sufficient to permit the components to cure to form the sustained release apparatus.

It has surprisingly been found that the use of the process according to the present invention permits preparation of a sustained release apparatus with significantly increased payloads.

As described above, the silicone base polymer may include a methyl-vinyl silicone polymer. The silicone base polymer may further include a reinforcing filler, e.g. a fumed silica. Fumed silica provides a high surface area relative to its weight so is preferred for high tear strength applications such as extrusion.

The process of preparing the sustained release apparatus is a multi-step process; e.g. pre-mix, mix, form, cure, and optionally coat. This permits the composition to be mixed thoroughly with silicone base polymer before the pharmaceutical active and catalyst are brought into contact.

Accordingly, pharmaceutical actives, e.g. sulfur containing chemicals, which heretofore could not be used, e.g. due to inhibition of silicone curing, may be used in the process according to the present invention.

By utilising a pre-mixing step, potential interference between the pharmaceutical active and catalyst may be reduced or minimized. The pre-mixing process also enables more thorough dispersion of the pharmaceutical actives and carriers without adding to the “work-time” of the final silicone mixture.

Surprisingly, temperatures between approximately 100° C. to 200° C., preferably approximately 100° C. to 150° C. may be used.

As the process may be conducted at, or below, approximately 200° C., the method may be applied to the preparation of delivery systems for pharmaceutical actives including sensitive, particularly heat-sensitive, pharmaceutical actives. The duration of the curing step may range from approximately 30 seconds to 180 minutes depending upon the type of process used. For heat-sensitive actives, a curing time of approximately 30 seconds to 30 minutes at a temperature below the degradation temperature, preferably approximately 30 seconds to 15 minutes, more preferably approximately 45 seconds to 5 minutes, may be used.

The catalyst used may be a peroxide or metal catalyst. However, pharmaceutical actives, e.g. sulfur-containing pharmaceuticals, which heretofore could not be used, e.g. due to fouling of the catalyst, may be used in the process according to the present invention.

Such curing conditions are preferably achieved utilising a metal catalyst, more preferably a platinum or rhodium catalyst.

A platinum-containing catalyst is preferred for medical applications. If a platinum catalyst is used, it may or may not be attached to an organic ligand. The preferred catalyst is dependent upon the choice of inhibitor, concentration of inhibitor, concentration of cross-linker, and the desired curing profile.

Preferably the platinum catalyst is present in amounts of from approximately 0.05% to 0.25%, by weight, based on the total weight of the reaction mixture.

The relatively high concentration of metal catalyst may compensate for the relatively low temperatures at which the process is conducted. For convenience, the metal catalyst may be provided in a mixture with a portion of the silicone base polymer component.

As the process according to the present invention is conducted at such relatively low temperatures, a curing inhibitor that will act as a curing inhibitor at such low temperatures is required. Preferably the low temperature curing inhibitor includes an unsaturated cyclosiloxane, more preferably tetramethyl tetravinyl cyclosiloxane.

The amount of inhibitor used is dependent on the curing temperature selected, the lower the temperature the lower the concentration of inhibitor required. A concentration of approximately 2.5 to approximately 15% by weight preferably approximately 5 to 10% may be used.

In a preferred form, where the pharmaceutically active component does not tend to inhibit the silicone curing process, a portion of the pharmaceutically active component may be included in the first part. This is preferred where a high loading capacity of active is to be achieved.

In a preferred embodiment of the process of the present invention, a carrier for the pharmaceutical active may be included. Accordingly, the process may further include

providing a carrier for the pharmaceutically active component in an amount of from approximately 15% to 25% by weight based on the total weight of the reaction mixture; and

pre-mixing the pharmaceutical carrier in the first part.

The pharmaceutical carrier may preferably include a sodium chloride, mannitol or a mixture thereof. injection-molding processes may utilize up to 100% liquid silicone base polymer. Compression-molding or transfer-molding may utilise approximately 0.5 to 20% by weight, preferably approximately 2.5 to 7.5% by weight of a liquid silicone component.

The cross-linking agent utilised in the process according to the present invention may be of any suitable type. A slioxane polymer; e.g. a partially methylated polyslioxane polymer, may be used. A short chain partially hydrogenated dimethyl siloxane polymer is particularly preferred.

The cross-linking agent may be present in amounts of from approximately 5% to 25% by weight, preferably approximately 10% to 15% by weight based on the total weight of the reaction mixture.

As stated above, the sustained release apparatus is preferably provided with a silicone coating. Accordingly in a preferred aspect of the present invention, the process may further include

providing a liquid coating composition; and

coating the apparatus with the coating composition.

The liquid coating composition may include a liquid silicone component, for example a liquid siloxane polymer.

The liquid coating composition may be applied utilising any standard technique. A dip coating process may be used, and the coating permitted to dry.

In a further preferred aspect of the present invention, the coating may be modified to provide a stronger coating layer and to extend the life of the implant. Accordingly, in this aspect, the process may further include

providing

a liquid coating composition including

a liquid silicone base material;

a cross-linking agent; and

metal catalyst

coating the apparatus with the coating composition; and

heating the coated apparatus to a temperature and for a time sufficient to cure the coating layer.

The liquid silicone base material of the coating composition may be an unsaturated silicone, e.g. siloxane polymer. The liquid silicone base material may be the same as, or similar to, the low temperature curing inhibition material described above. A tetramethyl tetravinyl cyclosiloxane may be used.

The liquid silicone base material may be present in the coating composition in amounts of from approximately 35% to 95% by weight, preferably approximately 40% to 80% by weight, more preferably approximately 50% to 70% by weight, based on the total weight of the coating composition.

The cross-linking agent of the coating composition may be a short chain liquid siloxane polymer. The cross-linking agent may be the same as, or similar to, the cross-linking agent described above. A short chain hydrogenated dimethyl polysiloxane is preferred.

The metal catalyst may be a platinum or rhodium catalyst, as described above.

The coating process may be run utilising a batch process or may preferably be conducted continuously with the formation of the apparatus. For example the coating process may be conducted utilising a co-extruslon apparatus, such that the coating layer may be delivered concentrically around the sustained release apparatus. The coating process may accordingly be conducted at temperatures and for times similar to those described above.

The cross-linking agent may be present in the coating composition in amounts of from approximately 2.5% to 25% by weight, preferably approximately 5% to 15% by weight, based on the total weight of the coating composition.

The sustained release apparatus of the present invention may have a rod-like shape, for example it is selected from circular cylinders, prisms, and elliptical cylinders. When the device will be administered using an injector-type instrument, a circular cylindrical device is preferred since the injector body and the injection needle typically have a circular cylindrical shape, though other shaped objects may be used. For example, dog microchips may be administered using an injector type instrument.

The size of the pharmaceutical formulation of the present invention may, in the case of subcutaneous administration, be relatively small. For example using an injector-type instrument, the configuration may be circular cylindrical, and the cross-sectional diameter in this embodiment is preferably approximately 0.5 to 4.0 mm, more preferably 0.5 to 1.7 mm, and the axial length is preferably approximately 1 to 40 mm, more preferably 10 to 30 mm.

The thickness of the outer layer should be selected as a function of the material properties and the desired release rate. The outer layer thickness is preferably 0.02 mm to 2 mm, more preferably 0.10 mm to 1 mm, and even more preferably 0.15 mm to 0.2 mm.

The ratio of the axial length of the pharmaceutical formulation to the cross-sectional diameter of the inner layer may, in any case, be one or more and is more preferably two or more and most preferably five or more.

Where a double-layer structure is used, the pharmaceutical-containing inner layer and the drug-impermeable outer layer may be fabricated separately or simultaneously. Silicone is known for swelling with water and being gas-permeable.

A pharmaceutical formulation with an open end at one terminal may be fabricated by dipping one terminal of the pharmaceutical formulation into a solution which dissolves the outer-layer material and drying it, or by covering one terminal end of the pharmaceutical formulation with a cap made from the outer-layer material. In addition, the fabrication may comprise insertion of the inner layer into an outer-layer casing with a closed-end at one terminal, which are separately produced, and also formation of the inner layer in said casing.

In a further aspect of the present invention there is provided a method for the therapeutic or prophylactic treatment of a disease condition in an animal (including a human) requiring such treatment, which method includes administering to the animal a sustained release apparatus including

a silicone support material; and

a pharmaceutically active composition carried in or on the silicone support material;

the pharmaceutically active composition including

at least one pharmaceutically active component; and optionally

a carrier therefor;

the pharmaceutically active component being present in amounts ranging from 30 to 75% by weight, preferably approximately 35% to 65% by weight, based on the total weight of the sustained release apparatus.

As stated above, it has been found that the pharmaceutical payload may be increased by the sustained release apparatus according to the present invention when compared to the prior art. Diseases which were heretofore untreatable may now be treated over an extended period of time utilising the apparatus of the present invention.

For example, in animals suffering from parasitic infections such as fleas, the animals may be treated utilising the sustained release apparatus including an anti-parasitic drug such as a macrocyclic lactone, e.g. ivermectin, moxidectin, eprinomectin, doramectin or mixtures thereof.

Heretofore, it was not possible to achieve a required blood concentration threshold to permit treatment of such a parasitic disease utilising a sustained release approach, as the required blood concentration threshold could not be achieved utilising such a mechanism.

In a further preferred form, the method according to this aspect of the present invention permits the treatment, over an extended period, of diseases and related indications heretofore not treatable due to the sensitivity of the pharmaceutical active.

In this form, the sustained release apparatus may take the form of a biocompatible article as described above, e.g. medical apparatus or implant, as silicone support material.

In an alternative embodiment a growth hormone, e.g. recombinant porcine somatotropin rPST may be administered to an animal. The required blood concentration may be maintained for an extended period.

The method of administration may include subcutaneous or intramuscular injection, intranasal insertion or indwelling intrarectal insertion or indwelling, for example as a suppository or utilising oral administration.

The animals to be treated may be selected from the group consisting of sheep, cattle, goats, horses, camels, pigs, dogs, cats, ferrets, rabbits, marsupials, buffalos, yacks, primates, humans, birds including chickens, geese and turkeys, rodents including rats and mice, fish, reptiles and the like.

The method according to the present invention is particularly applicable to larger animals, e.g. cattle, sheep, pigs, dogs and humans where high dosage levels are required to achieve the prerequisite threshold pharmaceutical active blood levels for successful treatment of selected disease indications.

The present invention will now be more fully described with reference to the accompanying figures and examples. It should be understood, however, that the description following is illustrative only and should not be taken in any way as a restriction on the generality of the invention described above.

In the figures:

FIG. 1 is a diagrammatic representation of an asymmetric covered rod design of a sustained release apparatus according to the present invention.

In the figure, the lighter colour illustrates a 100% silicone covering and the darker colour in the silicone carrier carrying the pharmaceutical active.

FIG. 2 is a diagrammatic representation of an eccentric covered rod design of a sustained release apparatus according to the present invention.

### EXAMPLE 1

rPST formulation 1

The A-part of a rPST formulation was prepared as follows.

First a platinum catalyst masterbatch (Pt MB) was prepared by mixing on a two-roll mill:

10.0 g silicone-base material (base 1) comprising a 60 durometer vinyl substituted dimethyl siloxane 0.02 g of a platinum catalyst

Then 2.00 g of the Pt MB was mixed with the following on a two-roll mill:

4.00 g of 60 durometer silicone-base material

4.00 g of a 50:50 sugar mixture (60 durometer base with confectioners sugar)

This completed the A-part of the PST formulation.

A B-part of the PST formulation was then prepared as follows:

First the following were mixed on a two-roll mill:

5.60 g of 60 durometer silicone-base material

0.30 g of Hydride MB (which contained 33% by weight hydride fluid)

0.20 g of an FDA approved red pigment

Then 4.00 g of recombinant porcine somatotropin RPST were mixed in on the mill.

Next, both the A and B-parts were separated into four balls of approximately 2.5 g each. Then one ball of A was combined with one ball of B on the two-roll mill. This was loaded into the mold. The four 5 g shots produced 8 implants. These implants were then coated with liquid silicone and left to cure overnight.

### EXAMPLE 2

rPST formulation 2

26.0 g of rPST was placed in a 50 ml beaker. Then the following were added:

1.565 g 60 durometer silicone-base material

0.10 g of Red 324 (an FDA approved red pigment)

0.025 g hydride concentrate (cross-linking agent).

0.5 g of Pt MB was added. This was blended directly on the two-roll mill and loaded into the mold. This produced two implants that were then coated with liquid silicone and left to cure overnight

### EXAMPLE 3

PST formulation 3

An A-part of a rPST formulation was prepared as follows:

A platinum catalyst master batch (PtMB) was prepared by mixing a silicone-base material comprising a 60 durometer vinyl substituted dimethyl siloxane with a platinum catalyst on a two-roll mill to form a PtMB containing 5% w/w platinum.

An Apart of the rPST formulation was formed by mixing the following components on a two-roll mill.

TABLE 1

| A-part component | % by weight |
|---|---|
| Salt (NaCl, ground and sieved) | 24.33 |
| Mannitol | 24.33 |
| Silicone base polymer | 46.34 |
| EX849 (40 durometer vinyl substituted dimethyl siloxane) | |
| PtMB CPO-085 (5% w/w platinum catalyst) | 5.00 |

A B-part of the rPST formulation was then prepared as follows:

TABLE 2

| B-part component | % by weight |
|---|---|
| Silicone base polymer | 12.08 |
| EX849 (40 durometer vinyl substituted dimethyl siloxane) | |
| Cross-linking agent | 16.67 |
| XL2 (short chain hydrogenated dimethyl polysiloxane | |
| Low temperature curing inhibitor | 11.67 |
| 88765 (tetramethyl tetravinyl cyclosiloxane) | |
| Low viscosity polymer* | 10.42 |
| (Silicone polymer viscosity modifier) | |
| Recombinant porcine somatotropin (rPST) | 49.17 |

*Low viscosity silicone polymer component added to reduce shear forces during extrusion.

The first four components are mixed together in a suitable container and the rPST subsequently added.

By including the RPST active in the B-side, inhibition of silicon curing is reduced or eliminated.

The A-part and B-part of the rPST formulation are mixed in a single screw co-extruder in a ratio of approximately 1 to 2. The single screw extruder has a L:D ratio of 1 to 20.

The process was conducted in a single screw co-extrusion apparatus where a coating layer is formed concentrically and coterminously with the apparatus.

Experiment 1A

The following ingredients were mixed to form a coating composition:

TABLE 3

| Coating layer component | % by weight |
|---|---|
| Liquid silicone base polymer | 93.5 |
| 88765 (tetramethyl tetravinyl cyclosiloxane) | |
| Cross-linking agent | 4.0 |
| XL2 (short chain hydrogenated dimethyl polysiloxane) | |
| Platinum catalyst | 2.5 |
| PtMB CPO-085 (5% w/w platinum catalyst) | |

All equipment is cooled to approximately 15° C.

The reaction mixture is then introduced into a IR curing oven at approximately 120° C. and the mixture passes through the oven in approximately 60 seconds. The extrusion profile exits at approximately 39° C., thus permitting full curing of the reaction mixture but reducing or eliminating degradation of the heat sensitive rPST active.

### EXAMPLE 4

Ivermectin (IVM)

The following components were mixed together:

38.0 g IVM

6.2 g NaDCA

3.3 g sucrose

Silicone fluid was added until the powder was fully wetted.

The following components were then added to the mixture:

9.00 g of liquid silicone fluid

0.45 g of hydride concentrate (cross-linking agent).

1.00 g of Violet pigment (FDA Approved).

0.5 g of Pt MB was spread out on the two-roll mill. 4.50 g of the mixture was gradually mixed into the Pt MB.

The titration was then begun as follows:

1. Spread 2.00 g of 40 dummeter FDA-grade silicone-base material

2. 49 g of B-side mixture was added.

3. 5.00 g of the resulting mixture was removed.

4. Repeat steps 1-3.

The above titration procedure was performed 11 times.

TABLE 4

| Titration | Concentration of | Concentration of | Result of |
|---|---|---|---|
| number | powder % | IVM % | formulation |
| 1 | 82.0 | 65.6 | Dry and crumbling |
| 2 | 78.8 | 63.0 | Too stiff |
| 3 | 75.5 | 60.4 | Too stiff |
| 4 | 72.1 | 57.7 | Stiff |
| 5 | 88.7 | 54.9 | Stiff |
| 6 | 65.2 | 52.1 | No problem |
| 7 | 61.5 | 49.2 | No problem |
| 8 | 57.8 | 46.2 | No problem |
| 9 | 54.0 | 43.2 | No problem |
| 10 | 50.0 | 40.0 | No problem |
| 11 | 45.8 | 36.6 | No problem |

After all 11 titrations were completed, each 5.00 g sample was mixed with 0.050 g of Pt MB on a two-roll mill. The first titration sample was not suitable for molding. The remaining samples were loaded into a transfer mold and two implants were made from each sample. Implants from samples 2 and 3 were of poor quality, but the remaining samples were excellent implants. The implants were then dip-coated with liquid silicone. All the implants produced from samples 2 to 11 were injected into animals for dinical studies.

### EXAMPLE 5

Four calves were implanted with 20×0.2 cm lengths of SDS IVM implants. Implants were placed subcutaneously in the ear.

Serum samples were analysed by the Australian Government Analytical Laboratories, Perth, Western Australia.

TABLE 5

| | Serum Ivermectin (ng/ml) | | | | |
|---|---|---|---|---|---|
| Calf number | Week 0 | Week 1 | Week 2 | Week 3 | Week 4 |
| 11 | ND | 1.0 | 0.25 | — | — |
| 17 | ND | 1.4 | 1.3 | 0.81 | 0.26 |
| 43 | ND | 1.8 | 2.0 | 1.8 | 1.9 |
| 55 | ND | 1.8 | 1.3 | 1.1 | 1.1 |

Abbreviations

ND=Less than 0.2 ng/ml

Calf number 11 had its implants removed one week after implantation. While calf number 17 had its implants removed at 4 weeks.

It will be understood that the invention disclosed and defined in this specification extends to all alternative combinations of two or more of the individual features mentioned or evident from the text or drawings. All of these different combinations constitute various alternative aspects of the invention.

It will also be understood that the term “comprises” (or its grammatical variants) as used in this specification is equivalent to the term “includes” and may be used interchangeably and should not be taken as excluding the presence of other elements or features.

1-77. Cancelled.

78. A sustained release apparatus including
a silicone support material; and
a pharmaceutically active composition carried in or on the silicone support material;
the pharmaceutically active composition including
at least one pharmaceutically active component; and optionally
a carrier therefor;
the pharmaceutically active component being present in amounts of from approximately 30% to 75% by weight, based on the total weight of the sustained release apparatus.

79. A sustained release apparatus according to claim 78, wherein the apparatus is of the uncovered or covered rod, or dispersed matrix type.

80. A sustained release apparatus according to claim 79, wherein the apparatus takes the form of a rod bearing a silicone coating thereover.

81. A sustained release apparatus according to claim 78, wherein the pharmaceutically active component is present in amounts of from approximately 40% to 50% by weight, based on the total weight of the apparatus.

82. A sustained release apparatus according to claim 78, wherein the silicone support material is formed from a silicone base polymer.

83. A sustained release apparatus according to claim 82, wherein the silicone base polymer includes a methyl-vinyl polysiloxane polymer.

84. A sustained release apparatus according to claim 82, wherein the silicone base polymer includes a silicone elastomer including a fumed silica as reinforcing filler.

85. A sustained release apparatus according to claim 82, wherein the silicone base polymer is present in amounts of from approximately 15% to 70% by weight, based on the total weight of the apparatus.

86. A sustained release apparatus according to claim 78, wherein the pharmaceutically active composition includes a pharmaceutically active component selected from one or more of the group consisting of acetonemia preparations, anabolic agents, anaesthetics, analgesics, anti-acid agents, anti-arthritic agents, antibodies, anti-convulsivants, anti-fungals, anti-histamines, anti-infectives, anti-inflammatories, anti-microbials, anti-parasitic agents, anti-protozoals, anti-ulcer agents, antiviral pharmaceuticals, behaviour modification drugs, biologicals, blood and blood substitutes, bronchodilators and expectorants, cancer therapy and related pharmaceuticals, cardiovascular pharmaceuticals, central nervous system pharmaceuticals, coccidiostats and coccidiocidals, contraceptives, contrast agents, diabetes therapies, diuretics, fertility pharmaceuticals, growth hormones, growth promoters, hematinics, hemostatics, hormone replacement therapies, hormones and analogs, immunostimulants, minerals, muscle relaxants, natural products, nutraceuticals and nutritionals, obesity therapeutics, ophthalmic pharmaceuticals, osteoporosis drugs, pain therapeutics, peptides and polypeptides, respiratory pharmaceuticals, sedatives and tranquilizers, transplantation products, urinary acidifiers, vaccines and adjuvants and vitamins.

87. A sustained release apparatus according to claim 86, wherein the pharmaceutically active component includes an anti-parasitic agent which includes ivermectin.

88. A sustained release apparatus including
a silicone support material; and
an anthelmintic composition carried in or on the support material;
the anthelmintic composition including
an anthelmintic component; and optionally
a carrier therefor;
the anthelmintic component being present in amounts of from approximately 30% to 75% by weight, based on the total weight of the apparatus.

89. A sustained release apparatus according to claim 88, wherein the anthelmintic component includes ivermectin.

90. A process for the preparation of a sustained release apparatus including
a silicone support material; and
a pharmaceutically active composition carried in or on the silicone support material;
the pharmaceutically active composition including
at least one pharmaceutically active component; and optionally
a carrier therefor;
the pharmaceutically active component being present in amounts of from approximately 30% to 75% by weight, based on the total weight of the sustained release apparatus, which process includes
providing
a silicone base polymer;
a cross-linking agent;
a pharmaceutically active component;
a peroxide or metal catalyst; and
a low temperature curing inhibitor;
pre-mixing at least a portion of the silicone base polymer and the metal catalyst together to form a first part;
pre-mixing the cross-linking agent, low temperature curing inhibitor, any remaining silicone base polymer, and pharmaceutical active for a time sufficient to at least partially wet the pharmaceutical active and form a second part; and
mixing the first and second parts together as a batch or continuously; and
feeding the mixture into a molding or extrusion apparatus at a relatively low temperature for a relatively short time sufficient to permit the components to cure to form the sustained release apparatus.

91. A process according to claim 90, wherein the silicone base polymer includes a methyl-vinyl siloxane polymer.

92. A process according to claim 90, wherein the silicone base polymer further includes a reinforcing filler.

93. A process according to claim 90, wherein the cross-linking agent includes a partially hydrogenated polysiloxane polymer.

94. A process according to claim 90, wherein the catalyst is a platinum catalyst present in amounts of from approximately 0.05% to 0.25%, by weight, based on the total weight of the reaction mixture.

95. A process according to claim 90, wherein the low temperature curing inhibitor includes a tetramethyl tetra-vinyl cyclosiloxane.

96. A process according to claim 90, further including
providing a carrier for the pharmaceutically active component in an amount of from approximately 15% to 25% by weight based on the total weight of the reaction mixture; and
pre-mixing the pharmaceutical carrier in the first part.

97. A process according to claim 96, wherein the pharmaceutical carrier includes sodium chloride or mannitol or mixtures thereof.

98. A process according to claim 90, wherein a portion of the pharmaceutically active component is included in the first part.

99. A process according to claim 90, further including
providing
a liquid coating composition including
a liquid silicone base material;
a cross-linking agent; and
metal catalyst
coating the apparatus with the coating composition; and
heating the coated apparatus to a temperature and for a time sufficient to cure the coating layer.

100. A biocompatible article including a sustained release apparatus including
a silicone support material; and
a pharmaceutically active composition carried in or on the silicone support material;
the pharmaceutically active composition including
at least one pharmaceutically active component; and optionally
a carrier therefor;
the pharmaceutically active component being present in amounts of from approximately 30% to 75% by weight, based on the total weight of the sustained release apparatus.

101. A biocompatible article according to claim 100, wherein the pharmaceutically active component is present in amounts from approximately 40% to 50% by weight, based on the total weight of the sustained release apparatus.

102. A biocompatible article according to claim 100, wherein the biocompatible article is a medical instrument, apparatus or prosthetic device or part thereof.

103. A biocompatible article according to claim 102, wherein the biocompatible article is a catheter, prosthetic appliance or medical implant for reconstructive dental or cosmetic surgery.

104. A biocompatible article according to claim 100, wherein the pharmaceutically active component includes an anti-coagulation agent an anti-anginal agent.

105. A method for the therapeutic or prophylactic treatment of a disease condition in an animal (including a human) requiring such treatment, which method includes administering to the animal a sustained release apparatus including
a silicone support material; and
a pharmaceutically active composition carried in or on the silicone support material;
the pharmaceutically active composition including
at least one pharmaceutically active component; and optionally
a carrier therefor;
the pharmaceutically active component being present in amounts ranging from 30 to 75% by weight, based on the total weight of the sustained release apparatus.

106. A method according to claim 105, wherein the pharmaceutically active component is present in amounts ranging from 40% to 50% by weight, based on the total weight of the apparatus.

107. A method according to claim 105, wherein the sustained release apparatus forms part of a biocompatible article.
